(19) United States
(12) Patent Application Publication
(10) Pub. No.: US 2015/0080701 A1
(43) Pub. Date: Mar. 19, 2015

(54) METHOD AND DEVICE TO EVALUATE A MEDICAL EXAMINATION

(71) Applicant: Siemens Aktiengesellschaft, Muenchen (DE)

(72) Inventors: Annemarie Hausotte, Erlangen (DE); Sebastian Schmidt, Weisendorf (DE)

(73) Assignee: Siemens Aktiengesellschaft, Muenchen (DE)

(21) Appl. No.: 14/490,973

(22) Filed: Sep. 19, 2014

(30) Foreign Application Priority Data
Sep. 19, 2013 (DE) 102013218800.5

(51) Int. Cl. G06T 7/00; G01R 33/56; A61B 8/08; A61B 6/00

(52) U.S. Cl. CPC G06T 7/0014; A61B 6/469; A61B 6/463; G01R 33/5608; A61B 8/5207; G06T 2207/10132; G06T 2207/10088; G06T 2207/10104; G06T 2207/30004
USPC 600/407; 382/128

(57) ABSTRACT

In a method and device to evaluate an examination of an examination subject with a medical imaging apparatus, an examination data set of the examination subject is imported into the evaluation device. A reference data set is associated with the imported examination data set, wherein the reference data set includes at least one reference region. At least one examination region of the examination data set is established by the evaluation device using the at least one reference region of the reference data set. The establishment of the at least one examination region of the examination data set can include a selection of at least one image of the examination data set; a marking of the at least one examination region in at least one image of the examination data set; and a segmentation of the at least one examination region in at least one image of the examination data set.

### BACKGROUND OF THE INVENTION

1. Field of the Invention

The present invention concerns: a method to evaluate an examination of an examination subject with a medical imaging apparatus, and a corresponding evaluation device and medical evaluation system; that enable execution of such a method as well as a non-transitory, computer-readable data storage medium encoded with programming instructions that cause a computer to execute such a method.

2. Description of the Prior Art

The evaluation of medical image data is a widespread field of application, in particular in clinical use.

In an everyday clinical environment, a suitable preparation of medical image data is desired, such as for an efficient and goal-oriented evaluation or diagnosis of an extremely complex problem, particularly if multiple medical image data are available.

### SUMMARY OF THE INVENTION

An object of the invention is to provide a method that facilitates an evaluation of such image data, and with which a presentation of an examination region (that is determined from the image data) is possible in a simple manner, depending on different boundary conditions.

A method in accordance with the invention to evaluate an examination of an examination subject with a medical imaging apparatus by operation of an evaluation unit, includes the following steps. An examination data set of the examination subject is imported into the evaluation unit. A reference data set is associated with the imported examination data set, the reference data set including at least one reference region. At least one examination region of the examination data set is established using the at least one reference region of the reference data set.

A medical imaging apparatus is an apparatus, such as an electronic and/or information technology apparatus, to acquire, process, evaluate and/or store image information in the form of image data. For example, acoustic methods such as ultrasound (US), emission methods such as emission computed tomography (ECT) and positron emission tomography (PET), optical methods, radiological methods such as x-ray tomography and computed tomography (CT) can be used to acquire the image information. The acquisition can also take place by magnetic resonance tomography (MR or MRT) or by combined modalities. The medical imaging apparatus can supply 2-dimensional (2D) or multidimensional (such as 3-dimensional (3D) or 4-dimensional (4D) image data) image data that can advantageously be stored and/or processed in different formats. The medical imaging apparatus can be used in diagnostics, for example in medical diagnostics.

The importation of an examination data set of the examination subject includes the importation of at least one image of the examination subject. Given an importation of more than one image, it is also possible that the images originate from different modalities, thus different medical imaging apparatuses. The examination subject advantageously comprises a patient.

The association of a reference data set with the imported examination data set includes the association of data of what is known as a standard patient with the examination subject. The standard patient is normally not the current examination subject to be examined. Rather, the standard patient includes information of a plurality of patients that are similar to the examination subject in a predetermined manner. For example, the similarity to the examination subject can be established by age, gender, height, existing prior and/or underlying illnesses, or additional features defined by a specialized medical personnel. For example, the standard patient can be represented by an average picture of this plurality of patients. The solution according to the invention includes the provision of multiple standard patients so that a representative standard patient can be associated for a plurality of examination subjects.

The reference data set of the standard patient also includes at least one reference region, i.e. a specially distinguished examination region that is advantageous to the evaluation of the examination. A position of the reference region—in particular a location and/or an extent of the reference region—within the standard patient is thereby considered to be known.

The invention utilizes this known reference region of the reference data set in order to establish at least one examination region of the examination data set, i.e. its position, namely its location and/or extent, within the examination subject.

In a preferred embodiment, the association of the reference data set with the imported examination data set takes place using information about the examination subject. For example, this information includes (but not exclusively) gender, age or a disease progression of the examination subject. For example, such information can also originate from DICOM (Digital Imaging and Communications in Medicine) attributes, thus attributes of an open standard to store and to exchange information in medical image data management. For instance, DICOM attributes include properties of a contrast or an orientation or other attributes such as, for instance, points in time of a measurement, information about a contrast agent administration or the gender of an examination subject. Different attributes can differ in various levels of scalability; for example, they can be nominally, ordinally or cardinally scalable. Via the wide spread of the DICOM standard, a high measure of compatibility is provided since the interoperability between systems of different manufacturers is possible. The association using this information also enables an optimized selection of a reference data set, i.e. a suitable selection of a standard patient matching the examination subject.

In an embodiment, the association of the reference data set with the imported examination data set includes a registration of the reference data set with the imported examination data set. As used herein, a registration is a process that brings the reference data set and the imported examination data set into agreement with one another, for example with regard to a viewing angle, a point in time or an acquisition position. The registration itself can be applied globally (i.e. predominantly to the complete data sets) or also locally (i.e. predominantly to defined regions of the data sets). The registration preferably occurs automatically, but it can also be started manually by a user. This facilitates a subsequent image evaluation since an at least partial reconciliation if the image data is made, and additionally saves time.

In a further embodiment, the at least one reference region of the reference data set is selected. For example, what is thereby to be understood by this selection is a manual selection of a reference region by a user, or also a manual or automatic selection of one or more reference regions from a predetermined list of reference regions. Criteria for such a selection can take into account a specific region to be examined of the examination subject or other framing conditions. This facilitates an identification of a defined examination region, and thus a subsequent image evaluation, and additionally saves time.

In another embodiment according to the invention, the establishment of the at least one examination region of the examination data set includes a selection of at least one image of the examination data set. If the examination data set includes more than one image—i.e. map or slice image of the examination subject, for example—it can be the case that the examination region of the examination data set that belongs to the reference region of the reference data set is not detectable in each of these images, or is not even imaged at all. In the embodiment according to the invention, an image is (preferably automatically) selected in which the examination region of the examination data set that belongs to the reference region of the reference data set is detectable. This facilitates a navigation through a plurality of image data and means a significant time savings for a user.

In a preferred embodiment, the establishment of the at least one examination region of the examination data set includes a marking of the at least one examination region in at least one image of the examination data set. In addition to the actual establishment of position (in particular location and/or extent) of an examination region within the examination subject, the solution according to the invention uses the direct marking of this examination region. As used herein a marking is, for example, a selection of a data matrix from the examination data set, or also a visual inking of the examination region in an image of the examination data set. This facilitates an identification of a defined examination region, and thus a subsequent image evaluation, and additionally saves time.

In another embodiment, the establishment of the at least one examination region of the examination data set includes a segmentation of the at least one examination region in at least one image of the examination data set. As used herein a segmentation is the generation of regions that are contiguous in terms of content via combination of adjacent image information (for example pixels or voxels) corresponding to a defined homogeneity criterion. The segmentation can take place automatically; however, at least one suggestion for a segmentation can also initially be generated, which segmentation must be confirmed by a user. Using the segmentation suggestion, a suggestion for a contouring can additionally also be generated, thus for an envelope of the contiguous region. Once again, this facilitates an identification of a defined examination region, and thus a subsequent image evaluation, and additionally saves time.

In a further embodiment, the reference data set includes a database, in particular a rule database. What is thereby to be understood by a rule database is a database that includes information about the reference data set, i.e. about the standard patient matching the examination subject. For example, this information includes information about at least one position of at least one organ relative to at least one anatomical landmark. Using this information, once again an examination region of the examination data set can then be concluded from the reference data set. The reference regions are then to be understood as calculation rules to obtain the examination regions; obtaining the examination regions thus follows the rules established in the database. However, the anatomical landmarks can also be determined automatically in that significant image regions are identified automatically. This alternative embodiment also facilitates an identification of a determined examination region, and thus a subsequent image evaluation, and additionally saves time.

Within the scope of the present invention, an evaluation unit is also provided to evaluate an examination of an examination subject with a medical imaging apparatus.

The evaluation unit thereby includes a processing unit and is designed to implement the following steps. An examination data set of the examination subject is imported into the processing unit. A reference data set is associated with the imported examination data set by the processing unit, the reference data set including at least one reference region. At least one examination region of the examination data set is established using the at least one reference region of the reference data set by the processing unit.

In addition to, a medical evaluation system according to the invention has an evaluation unit as described above and at least one medical imaging apparatus.

The present invention also encompasses a non-transitory, computer-readable data storage medium encoded with programming instructions that, when the storage medium is loaded into a computerized processor, cause the processor to implement the method according to the invention as described above. The programming instructions may require program items (for example libraries and auxiliary functions) in order to realize the corresponding embodiments of the method. The programming instructions can be a source code that must still be compiled and linked or that only must be interpreted, or an executable software code that has only to be loaded into the corresponding computer for execution.

The electronically readable storage medium can be, for example, a DVD, a magnetic tape or a USB stick on which is stored electronically readable control information, in particular software.

The advantages of the evaluation unit according to the invention, and the medical evaluation system according to the invention, and the electronically readable storage medium according to the invention, essentially correspond to the advantages of the method according to the invention as described above. Features, advantages and alternative embodiments that are described with regard to the method apply as well to the other aspects of the invention.

### BRIEF DESCRIPTION OF THE DRAWINGS

FIG. 1 schematically illustrates a medical evaluation system according to the invention.

FIG. 2 is a flowchart of an embodiment of the method according to the invention.

FIG. 3 shows an example of an establishment of an examination region using a reference region in accordance with the invention.

### DESCRIPTION OF THE PREFERRED EMBODIMENTS

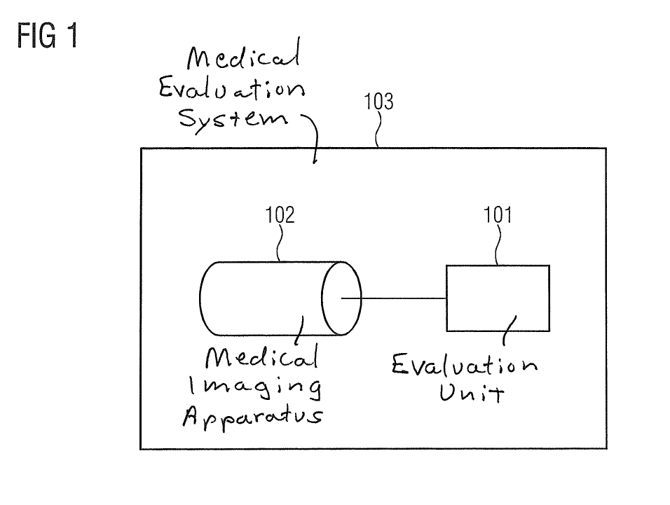

FIG. 1 shows a medical evaluation system 103 according to the invention. The medical evaluation system 103 has an evaluation unit 101 and a medical imaging apparatus 102 and is designed to evaluate an examination of an examination subject.

The medical imaging apparatus 102 is executed here as a magnetic resonance apparatus. Alternatively, the medical imaging apparatus 102 can also has a combined magnetic resonance positron emission tomography apparatus or additional medical imaging apparatuses 102 that appear to be reasonable to those skilled in the art.

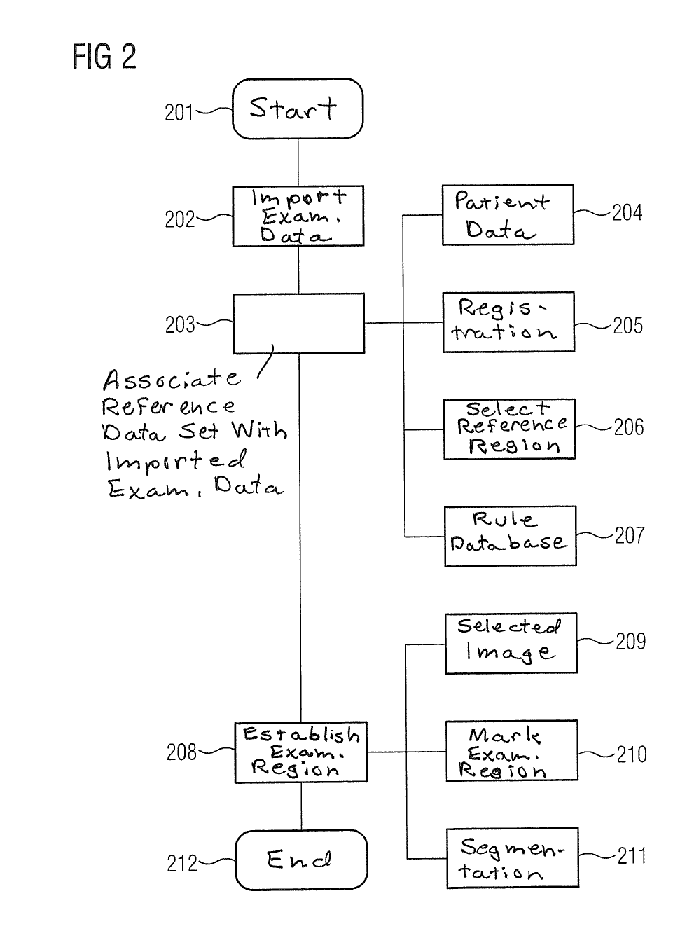

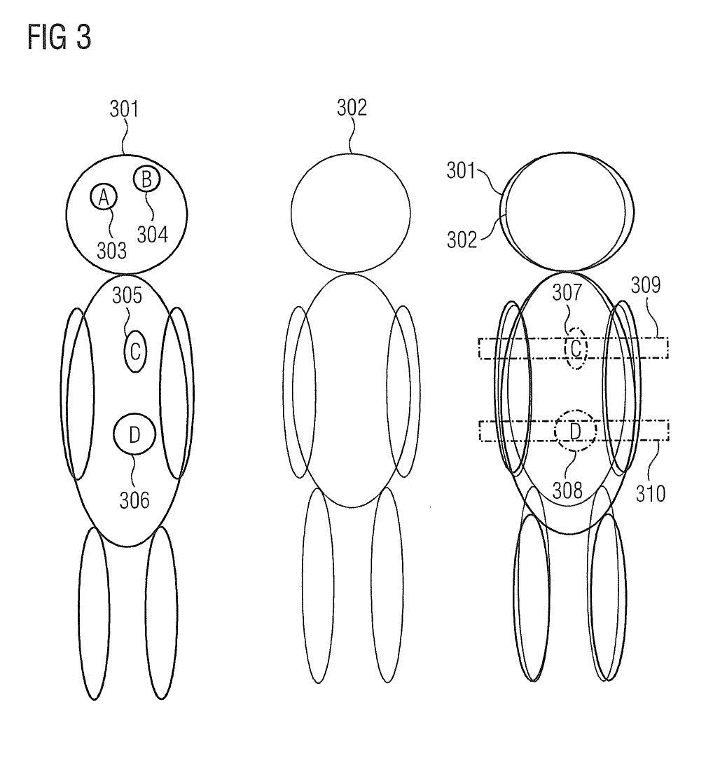

FIG. 2 shows a flowchart of an embodiment of the method according to the invention. The method includes the method steps 201 through 212, wherein description portions including the corresponding reference characters introduced in connection with FIGS. 1 and 3 are also used in the description of method steps 201 through 212.

The method steps 201 through 212 are implemented by the evaluation unit 101 of the medical evaluation system 103.

A first method step 201 identifies the start of an evaluation of an examination of an examination subject with the medical imaging apparatus 102 by the evaluation unit 101.

An importation of an examination data set 302 of the examination subject takes place in method step 202. The importation includes the importation of at least one image of the examination subject. Given an importation of more than one image, it is also possible that the images originate from different modalities (thus different medical imaging apparatuses 102).

An association of a reference data set 301 with the imported examination data set 302 takes place during a method step 203, wherein the reference data set 301 includes at least one reference region 303, 304, 305, 306 as illustrated in FIG. 3, for example using four reference regions 303, 304, 305, 306. The association includes the association of data of what is known as a standard patient with the examination subject. The standard patient is normally not the current examination subject to be examined. The reference data set of the standard patient also includes the at least one reference region 303, 304, 305, 306, i.e. a particularly distinguished examination region that is advantageous to the evaluation of the examination. A position of the reference region 303, 304, 305, 306 (i.e. a location and an extent of the reference region 303, 304, 305, 306) within the standard patient is considered to be known.

The association of the reference data set 301 with the imported examination data set 302 takes place in method step 204 using information about the examination subject. For example, this information includes (but not exclusively) gender, age or a disease progression of the examination subject. For example, such information can also originate from DICOM (Digital Imaging and Communications in Medicine) attributes, thus attributes of an open standard for storage and exchange of information in medical image data management. For instance, DICOM attributes include properties of a contrast or an orientation or other attributes, for instance points in time of a measurement, information about an administration of contrast agent or the gender of an examination subject. Different attributes can differ as in levels of scalability; for example, they can be nominally, ordinally or cardinally scalable.

In method step 205, the association of the reference data set 301 with the imported examination data set 302 includes a registration of the reference data set 301 with the imported examination data set 302. The registration itself can be applied globally (i.e. predominantly to the complete data sets) or also locally (i.e. predominantly to specific regions of the data sets). The registration preferably occurs automatically, but can also be started manually by a user.

During a method step 206, the at least one reference region 303, 304, 305, 306 of the reference data set 301 is selected. This selection is, for example, is a manual selection of a reference region 303, 304, 305, 306 by a user, or also a manual or automatic selection of one or more reference regions 303, 304, 305, 306 from a predetermined list of reference regions 303, 304, 305, 306.

In method step 207, the reference data set 301 includes a database, in particular a rule database. What is thereby to be understood by a rule database is a database that includes information about the reference data set 301, i.e. about the standard patient matching the examination subject. For example, this information includes information about at least a position of at least one organ relative to at least one anatomical landmark. Once again, an examination region 307, 308, 309, 310 of the examination data set 302 can be concluded from the reference data set 301 using this information. The reference regions 303, 304, 305, 306 are then to be understood as calculation rules to obtain the examination regions 307, 308, 309, 310. However, the anatomical landmarks can also be inherently determined automatically in that significant image regions are automatically identified.

In the embodiment of the method according to the invention, the method steps 204 through 207 can be used alternatively or can also be arbitrarily combined with one another.

In method step 208, an establishment of at least one examination region 307, 308, 309, 310 of the examination data set 302 takes place using the at least one reference region 303, 304, 305, 306 of the reference data set 301.

During a method step 209, the establishment of the at least one examination region 307, 308, 309, 310 of the examination data set 302 includes a selection of at least one image of the examination data set 302. If the examination data set 302 includes more than one image (i.e. for example map or slice image of the examination subject), it can be the case that the examination region 307, 308, 309, 310 of the examination data set 302 that belongs to the reference region 303, 304, 305, 306 of the reference data set 301 is not detectable in each of these images or is not even imaged. In this exemplary embodiment, an image is (advantageously automatically) selected in which the examination region 307, 308, 309, 310 of the examination data set 302 that belongs to the reference region 303, 304, 305, 306 of the reference data set 301 is detectable.

In method step 210, the establishment of the at least one examination region 307, 308, 309, 310 of the examination data set 302 includes a marking of the at least one examination region 307, 308, 309, 310 in at least one image of the examination data set 302. In addition to the actual establishment of position (thus location and extent) of an examination region 307, 308, 309, 310 within the examination subject, this examination region 307, 308, 309, 310 is thus marked directly.

In the method step 211, the establishment of the at least one examination region 307, 308, 309, 310 of the examination data set 302 includes a segmentation of the at least one examination region 307, 308, 309, 310 in at least one image of the examination data set 302. The segmentation can thereby take place automatically, but a suggestion for a segmentation can also initially be generated that must be confirmed by a user, for example by means of an input unit of the evaluation unit 101. In addition to this, a suggestion for a contouring—thus for an envelope of the region to be segmented—can also be generated using the segmentation proposal.

In the embodiment of the method according to the invention, the method steps 209 through 211 can alternatively be used or can also be arbitrarily combined with one another.

A last method step 212 identifies the end of the evaluation of an examination of an examination subject with a medical imaging apparatus 102 by means of an evaluation unit 101.

FIG. 3 shows an example of an establishment of an examination region 307, 308, 309, 310 using a reference region 303, 304, 305, 306.

Initially, an examination data set 302 of an examination subject is imported in method step 202. The examination data set 302 here is shown in the form of an image of an examination subject. The importation takes place by means of an evaluation unit 101.

In a next step (method step 203), a reference data set 301 is associated with the imported examination data set 302, wherein the reference data set 301 includes four reference regions 303, 304, 305, 306. The reference data set here is shown in the form of an image of a standard patient. The standard patient includes information of a plurality of patients that are similar to the examination subject in a predetermined manner. For example, the similarity to the examination subject can be established by age, gender, height, existing prior and/or underlying illnesses or additional features defined by a specialist medical personnel. For example, the standard patient can be represented by an average image of this plurality of patients.

In method step 208, at least one examination region 307, 308, 309, 310 of the examination data set 302 can now be established using the four reference regions 303, 304, 305, 306 of the reference data set 301. In the example present in the Figure, two examination regions 307, 308 are established since only a torso of the examination subject should be considered.

This association is presented in two different ways in FIG. 3.

For example, it can be realized via specification of an area of an examination region 309, 310. Within this region, a user can ultimately define an examination region 307, 308, for example via a marking in the image of the examination subject.

However, the association can also be realized directly via a transfer of the reference regions 305, 306 to the associated examination regions 307, 308 in that the images of examination subject and standard patient are superimposed on one another and the positions of the examination regions 307, 308 are determined according to the positions of the reference regions 305, 306.

In summary, the invention concerns a method to evaluate an examination of an examination subject with a medical imaging apparatus by means of an evaluation unit, including the following steps:
import an examination data set of the examination subject,
associate a reference data set with the imported examination data set, wherein the reference data set includes at least one reference region, and
establish at least one examination region of the examination data set using the at least one reference region of the reference data set.

In an embodiment, the establishment of the at least one examination region of the examination data set includes: a selection of at least one image of the examination data set; a marking of the at least one examination region in at least one image of the examination data set; and a segmentation of the at least one examination region in at least one image of the examination data set.

Although modifications and changes may be suggested by those skilled in the art, it is the intention of the inventor to embody within the patent warranted hereon all changes and modifications as reasonably and properly come within the scope of his contribution to the art.

We claim as our invention:

1. A method to evaluate an examination of an examination subject that was conducted with a medical imaging apparatus, comprising:
importing examination data, acquired from an examination subject with a medical imaging apparatus, into a computerized evaluation unit;
in said evaluation unit, associating a reference data set with the imported examination data set, said reference data set comprising at least one reference region; and
in said evaluation unit, establishing at least one examination region of the examination data set using said at least one reference region of the reference data set.

2. A method as claimed in claim 1 comprising associating said reference data set with said imported reference data set using information describing the examination subject.

3. A method as claimed in claim 1 comprising associating the reference data set with the imported examination data set by implementing a registration of the reference data set with the imported examination data set.

4. A method as claimed in claim 1 comprising, in said evaluation unit, selecting said at least one reference region of the reference data set.

5. A method as claimed in claim 1 comprising establishing said at least one examination region of said examination data set by a selection of at least one image of said examination data set.

6. A method as claimed in claim 1 comprising establishing said at least one examination region of said examination data set by marking said at least one examination region in at least one image of said examination data set.

7. A method as claimed in claim 1 comprising establishing said at least one examination region of the examination data set by segmentation of said at least one region in at least one image of said examination data set.

8. A method as claimed in claim 1 wherein said reference data set comprises a database.

9. A computerized evaluation unit to evaluate an examination of an examination subject that was conducted with a medical imaging apparatus, comprising:
a processor having an input into which examination data, acquired from an examination subject with a medical imaging apparatus, are imported into the processor;
said processor being configured to associate a reference data set with the imported examination data set, said reference data set comprising at least one reference region; and
said processor being configured to establish at least one examination region of the examination data set using said at least one reference region of the reference data set.

10. A medical evaluation system comprising:
a medical imaging apparatus in which an examination subject is situated, said medical imaging apparatus being configured to acquire examination data from the examination subject in the medical imaging apparatus;
a computerized evaluation unit into which said examination data are imported;
said evaluation unit being configured to associate a reference data set with the imported examination data set, said reference data set comprising at least one reference region; and
said evaluation unit being configured to establish at least one examination region of the examination data set using said at least one reference region of the reference data set.

11. A non-transitory, computer-readable data storage medium encoded with programming instructions, said data storage medium being loaded into a computerized processor and said programming instructions causing said processor to:
receive examination data, acquired from an examination subject with a medical imaging apparatus;
associate a reference data set with the imported examination data set, said reference data set comprising at least one reference region; and
establish at least one examination region of the examination data set using said at least one reference region of the reference data set.
